(19) United States
(12) Patent Application Publication
(10) Pub. No.: US 2008/0306146 A1
(43) Pub. Date: Dec. 11, 2008

(54) Dosing Regimens for Cox-2 Inhibitor

(75) Inventors: Kyung-Min Lim, Gyeonggi-do (KR); Mi-Young Park, Gyeonggi-do (KR); Kl-Wha Lee, Seoul (KR); Sung-ll Kim, Gyeonggi-do (KR); Shin Chung, Gyeonggi-do (KR)

Correspondence Address: Baker & Hostetler LLP;Attn: Jim Coffman, 45 Rockefeller Plaza, New York, NY, 10111, US

(73) Assignee: AMOREPACIFIC CORPORATION, Seoul (KR)

(21) Appl. No.: 12/092,304

(22) PCT Filed: Oct. 31, 2006
(86) PCT No.: PCT/KR2006/004481

§ 371 Date: May 1, 2008

Related U.S. Application Data
(60) Provisional application No. 60/732,052, filed on Nov. 2, 2005.

(51) Int. Cl. A61K 31/341; A61P 29/00; A61P 19/02; A61P 25/00; A61P 35/00

(52) U.S. Cl. 514/471

(57) ABSTRACT

The present invention relates to a potent cyclooxygenase-2 inhibitor and dosing regimens thereof to safely treat cyclooxygenase-2 mediated disorders.

### FIELD OF INVENTION

The present invention relates to a potent cyclooxygenase-2 inhibitor and dosing regimens thereof to safely treat cyclooxygenase-2 mediated disorders.

### BACKGROUND OF THE INVENTION

Cyclooxygenases are enzymes involved in the transformation of arachidonic acid into a variety of prostaglandins and thromboxanes. To date, there are at least two kinds of cyclooxygenases discovered. Cyclooxygenase-1 (COX-1) is constitutively expressed in a variety of tissues including the gastro-intestinal (GI) mucosa and the kidney. COX-1 is believed to be responsible for the maintenance of the homeostasis, for example, in the GI tract. Inhibition of COX-1 is known to be associated with the undesirable toxicity of perforation, ulceration and bleeding in the GI tract. In the meantime, cyclooxygenase-2 (COX-2) is induced upon inflammatory stimuli and known to be involved in the pathogenesis of inflammation and inflammation-associated disorders. Physiological and clinical aspects of COX-2 inhibitors have been reviewed from diverse perspectives. It is noted that the therapeutic scope of COX-2 inhibitors may comprise not only inflammation and inflammation-associated disorders including but not limited to osteoarthritis, rheumatoid arthritis, gouty arthritis, and ankylosing spondylitis, but also other COX-2 mediated disorders including but not limited to cancers and Alzheimer's disease. [Expert Opin. Ther. Patents vol 15, 9-32 (2005); Pharmacol. Rev. vol 56, 387-437 (2004); Nature Rev. Drug Discovery vol 2, 879-890 (2003)]

Traditional non-steroidal anti-inflammatory drugs (NSAIDs), such as aspirin, naproxen, ibuprofen, piroxicam, diclofenac, and so on, inhibit both COX-1 and COX-2 at therapeutically relevant exposure. Even though traditional NSAIDs have been widely used over a century to treat inflammation and inflammation-associated disorders, the notorious life threatening GI toxicity of traditional NSAIDs has posed big safety concerns in the use of traditional NSAIDs for the treatment of osteoarthritis, rheumatoid arthritis, gouty arthritis, low back pain, migraines, post-operative pains, cancer pain, menstrual pain, ankylosing spondylitis, tendinitis, dental pain, and so on.

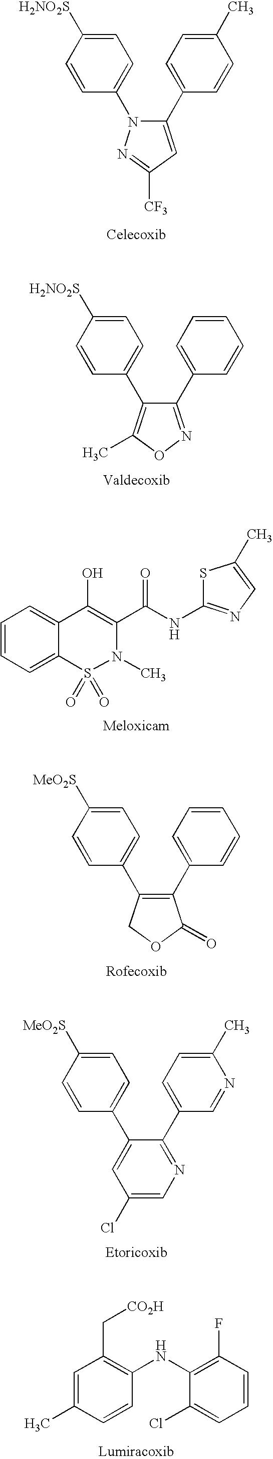

In order to reduce the notorious GI toxicity from inhibition of COX-1, selective COX-2 inhibitors have been extensively studied. To date, a variety of selective COX-2 inhibitors, are now or used to be available for clinical use, which include celecoxib, rofecoxib, valdecoxib, etoricoxib, lumiracoxib, and meloxicam. Clinical evaluation with selective COX-2 inhibitors clearly demonstrated improved GI safety compared to traditional NSAIDs. [N. Engl. J. Med. vol 343, 1520-1528 (2000); JAMA vol 284, 1247-1255 (2000); Lancet vol 364, 675-684 (2004)] Even though selective COX-2 inhibitors are regarded superior to traditional NSAIDs in the GI safety, the GI adverse events from use of selective COX-2 inhibitors may become more pronounced in susceptible populations such as elderly patients and patients on anti-tumor therapy. [EJC Suppl. vol 2, 14-20 (2004); JAMA vol 289, 2816-2819 (2003)] Furthermore, in vivo findings suggest that COX-2 upregulation plays a crucial role in adaptive cytoprotection against mild irritation in the stomach. [J. Clin. Enterol. vol 25, S105-S110 (1997); Br. J. Pharmacol. vol 123, 927-935 (1998)] Other findings suggest that COX-2 may be important for the wound healing in the GI tract. [J. Clin. Enterol. vol 27, S28-S34 (1998)] Thus, chronic inhibition of the COX-2 in the GI tract could lead to unwanted adverse events in the GI tract. COX-2 needs to be inhibited for anti-inflammatory and analgesic effect, though. It is desired to minimize the inhibition of the COX-2 in the GI tract for the GI safety.

Analyses of clinical data with selective COX-2 inhibitors suggested that use of selective COX-2 inhibitors might be associated with an increase in the cardiotoxicity compared to use of a traditional NSAIDs. [JAMA vol 286, 954-959 (2001)] Unlike traditional NSAIDs, selective COX-2 inhibitors lack the anti-thrombotic effect from the COX-1 inhibition in the platelet, which could account for a potential increase in the cardiotoxicty by use of a selective COX-2 inhibitor. Recently, rofecoxib 25 mg/day was found to be associated with an increased risk of thromboembolic cardiovascular events in a long term placebo-controlled cancer prevention trial (APPROVe). celecoxib at 400 mg/day and 800 mg/day was also associated with increases in the thromboembolic events in a long term placebo-controlled cancer prevention trial (APC). Furthermore, acute use of valdecoxib was found to be associated with an increase in the thromboembolic events in patients undergone coronary artery bypass graft (CABG). [Biocentury vol 13, No 4, A1-A4 (2005)] These findings indicate that inhibition of COX-2 is likely to result in an increase in cardiovascular toxicity, and that COX-2 inhibition is not desired for cardiovascular safety. It is noted that even traditional NSAIDs do not appear to be free of cardiovascular toxicity. Chronic administration of naproxen 500 mg in subjects at increased risk of Alzheimer's disease led to an increase in the cardiovascular toxicity in a placebo-controlled trial named ADAPT.

Given that hypertension is a well-established risk factor for thromboembolic events, [Lancet vol 335, 827-838 (1990)] improvement in the renal safety would be useful to reduce the potential cardiotoxicity of a selective COX-2 inhibitor. Frequent renal adverse events from use of a COX-2 inhibitor are sodium retention, edema and the resultant hypertension. COX-2 has been reported to be expressed in various parts of the kidney, such as the renal vasculature and macula densa, a tubular segment important for sensing the vascular tone and the release of renin. As clinical data accumulate on selective COX-2 inhibitors, NSAIDs and selective COX-2 inhibitors are taken as similar with regard to renal sodium excretion and glomerular filtration rate (GFR). [J. Pharmacol. Exp. Therapeut. vol 289, 735-741 (1999); J. Pain Symptom Management vol 23 (4S1), S15-S20 (2002)] Inhibition of the renal COX-2 would have contributed significantly to the observed cardiotoxicity of rofecoxib and celecoxib in the long term clinical trials. [Cleveland Clinic J. Med. vol 71 849-856 (2004)] It is desired not to inhibit COX-2 in the kidney for cardiovascular safety.

The COX-2 expressed in blood vessels (especially endothelial cells) produces vasodilatory prostacyclin (prostaglandin 12). Prostacyclin was also known locally to inhibit the prothrombotic activity of the platelets and to act on vascluar smooth muscle cells leading to vasodilation. [Expert Rev. Mol. Med. vol 5, 1-18 (2003)] The IP receptor (prostacyclin receptor) deficient mice were susceptible to vascular proliferation (i.e. vascular occlusion) and platelet activation in response to a vascular injury, suggesting that a reduction in the local level of prostacyclin may increase the risk of atherosclerosis as well as thromboembolic events. [Science vol 296, 539-541 (2002)] The observed acute increase in the cardiotoxicity from the acute use of valdecoxib in patients with CABG could be from an overt inhibition of the COX-2 in the blood vessels. [Circulation vol 111, 249 (2005)] There have been reports suggesting that COX-2 could be induced in vascular smooth muscle cells in response to vascular atherosclerotic stimuli, such as denudation of the endothelial layer, angiotensin II, high density lipoprotein (HDL), platelet-derived growth factor, and so on. [Biochem. Biophys. Res. Commun. vol 224, 808-814 (1996); Hypertension vol 35, 68-75 (2000); Br J. Pharmacol. vol 131, 1546-1552 (2000); Arterioscler. Thromb. Vasc. Biol. vol 19, 2405-2411 (1999)] COX-2 expressed in vascular smooth muscle cells in response to such pathogenic stimuli is expected to show cardiovascular protective roles including vasodilation and inhibition of proliferation and hypertrophy of vascular smooth muscle cells. COX-2 needs to be inhibited for the anti-inflammatory and analgesic effect in tissues of therapeutic concern, however, the inhibition of COX-2 in the blood vessels should be minimized for the cardiovascular safety.

In order for a selective COX-2 inhibitor with a short elimination half-life to maintain therapeutic effect until the next dosing, its Cₘₐₓ (i.e. maximum plasma concentration) needs to be high enough to yield a Cₘᵢₙ (i.e. minimum plasma concentration) in excess of the minimally required therapeutic concentration. A COX-2 inhibitor with a plasma elimination half life of 8 hours, for example, needs to possess a Cₘₐₓ in excess of the minimally required therapeutic concentration by about 8-fold (i.e. 2×2×2=8 times), if the COX-2 inhibitor is to be administered daily once without adopting a formulation of sustained release. As seen in the case of valdecoxib in patients with CABG, such a high Cₘₐₓ may not be desired for cardiovascular toxicity.

It is noted that the cardiotoxicity of COX-2 inhibitors appeared to increase with dose as seen in the cases for celecoxib and valdecoxib, [Biocentury vol 13, No 4, A1-A4 (2005); Circulation vol 111, 249 (2005)] suggesting that the cardiotoixity may increase with increase of COX-2 inhibition in tissues of cardiovascular safety concern. Maintaining COX-2 inhibition to a lowest level possible in tissues of cardiovascular safety concern would be of prime importance for cardiovascular safety in treating a COX-2 mediated disorder requiring a long term treatment with a COX-2 inhibitor, since the cardiotoxicity of a COX-2 inhibitor appears to increase with treatment duration. Therefore, chronic indications including but not limited to osteoarthritis and rheumatoid arthritis need to be managed with a COX-2 inhibitor at its lowest effective dose.

Prior art WO 00/61571 provided a novel class of COX-2 inhibitors represented by Formula A with 3(2H)furanone as a scaffold or pharmacophore for potent selective inhibition of COX-2 over COX-1,

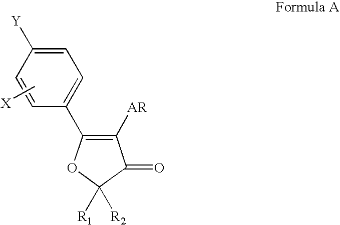

wherein:
X represents halo, hydrido, or alkyl;
Y represents alkylsulfonyl, aminosulfonyl, alkylsulfinyl, (N-acylamino)-sulfonyl, (N-alkylamino)sulfonyl, or alkylthio;
R₁ and R₂ are selected independently from lower alkyl radicals, or form a 4- to 6-membered aliphatic or heterocyclic group, taken together with the 2-position carbon atom of 3(2H)-furanone ring; and
AR represents a substituted or non-substituted aromatic group of 5 to 10 skeletal atoms.

Compounds of Formula A are selective COX-2 inhibitors with strong anti-inflammatory and analgesic activities in animal models, as demonstrated in the prior art WO 00/61571 and the literature. [J. Med. Chem. vol 47, 792-804 (2004)] For example, 5-{4-(aminosulfonyl)phenyl}-2,2-dimethyl-4-(3-fluorophenyl)-3(2H)furanone (Compound A of this invention) showed an ED₅₀ of 0.1 mg/kg/day by adjuvant-induced arthritis in male Lewis rats, whereas an ED₅₀ of 0.3 mg/kg/day was observed with positive comparator indomethacin. In carrageenan-induced thermal hyperalgesia in male SD rats, ED₅₀'s of 0.25 mg/kg and ˜1.0 mg/kg were observed for orally administered Compound A and indomethacin, respectively. Given that adjuvant-induced arthritis simulates well the pathogenic situations of human arthritis, a selective COX-2 inhibitor showing a strong potency by adjuvant-induced arthritis would show therapeutic activity at a small daily dose for the treatment of osteoarthritis and rheumatoid arthritis.

### SUMMARY OF THE INVENTION

This invention provides embodiments relating to dosing regimens for Compound A, by which safety benefits may be achieved in treating a subject with a disorder or disease mediated through COX-2.

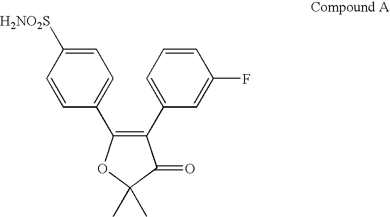

In one embodiment of the present invention, capsule drug products containing 0 to 15 mg of Compound A and appropriate amounts of excipients are provided.

In another embodiment, pharmacokinetic data in human subjects are presented following a single oral dose of Compound A at therapeutically relevant dose levels.

In yet another embodiment, are provided dosing regimens of loading dose and daily maintenance dose by which plasma levels of Compound A may reach the steady state levels essentially within the first day of dosing, maintained throughout the rest of maintenance dosing period, and decay thereafter with a pharmacokinetic profile as expected from the cases of the single dose administrations.

Compound A can be converted into a pharmaceutically-acceptable salt by neutralizing the compound, depending on the presence of an acidic group or a basic group in the compound, with an equivalent amount of an appropriate pharmaceutically-acceptable acid or base, such as potassium hydroxide, sodium hydroxide, hydrochloric acid, methansulfonic acid, citric acid, and the like. Compound A or a pharmaceutically-acceptable salt thereof can be administered along with various pharmaceutically-acceptable adjuvant ingredients, including but not limited to, citric acid, sodium chloride, tartaric acid, stearic acid, starch, gelatin, talc, sesame oil, ascrobic acid, methylcellulose, sodium carboxymethylcelluose, polyethyleneglycol (PEG), polypropyleneglycol, sweeteners, preservatives, water, ethanol, titanium oxide, sodium bicarbonate, silicified microcrystalline cellulose, soybean lecithin, and the like. Compound A or a pharmaceutically-acceptable salt thereof can be formulated in a variety of dosage forms, including but not limited to, tablet, powder, granule, hard capsule, soft capsule, and the like. Compound A or a pharmaceutically-acceptable salt thereof may be administered to a subject at a daily dose of up to several mg/kg body weight depending on the indications, symptoms, or conditions of the subject.

### Preparation of Capsule Drug Products

Quantities of Compound A were formulated with a diverse compositions of pharmaceutically acceptable excipients to manufacture capsule drug products containing 0 to 15 mg of Compound A as the active ingredient. Exemplary compositions of such capsule drug products are provided in Table 1. However, provision of such exemplary compositions is not intended to limit the compositions of the capsule drug products employed in the instant invention as provided in Table 1. The capsule drug products were manufactured by widely adopted methods with due controls including the drug strength, homogeneity, and dissolution profile.

TABLE 1
Compositions of Capsule Drug Products containing Compound A

| Ingredient | Quantity (per capsule) | | Function |
|---|---|---|---|
| Compound A | 0~15 | mg | Active Ingredient |
| Microcrystalline | 135~200 | mg | Filler & Diluent |
| Cellulose or Silicified | | | |
| Microcrystalline | | | |
| Cellulose | | | |
| Talc | 0~2 | mg | Glidant |
| Total Fill Weight | 150~200 | mg | |

| | | |
|---|---|---|
| Hard Gelatin Capsule | 1 (EA) | Capsule Shell |

### Pharmacokinetic Data in Healthy Male Subjects (Single Dose)

Capsule drug products of the present invention were evaluated in healthy male subjects for pharmacokinetic profiles following a single oral administration of capsule(s) containing a designated amount of Compound A. In Table 2, are summarized observed plasma pharmacokinetic data following a single oral administration. According to Table 2, Cₘₐₓ increased essentially dose proportionally. Long terminal elimination half-lives of 77 to 135 hours were observed on average, although there were some variations in the half-life.

TABLE 2
Pharmacokinetic data in healthy human male subjects
following a single oral administration of capsule
drug product(s) containing Compound A.

| Group | Dose | Cₘₐₓ(plasma) | Terminal Elimination Half-life |
|---|---|---|---|
| 1 | 1 mg | 1.65 ng/ml | — |
| 2 | 5 mg | 7.82 ng/ml | 118 ± 66 Hours |
| 3 | 8 mg | 13.3 ng/ml | 77 ± 8 Hours |
| 4 | 12 mg | 18.1 ng/ml | 135 ± 24 Hours |

### Dosing Regimens for Multiple Administrations

A long half life would be of advantage for a COX-2 inhibitor in that steady state plasma drug concentrations could be maintained without much fluctuation between doses by adopting a proper combination of loading and daily maintenance dose. For a drug with an elimination half-life of 5 days, for example, the plasma level of the drug in 24 hours would decrease only by less than 15%. As discussed above little difference between Cₘₐₓ and Cₘᵢₙ is advantageous to cardiovascular safety for a COX-2 inhibitor.

By assuming an elimination half-life of 4 to 5 days for Compound A, were designed multiple dosing regimens by which steady state plasma concentrations of Compound A are rapidly reached and maintained throughout the dosing period. Such dosing regimens are expected to be advantageous to the cardiovascular safety, in that the plasma levels of drug can be maintained to necessary levels without going high to overtly inhibit COX-2 in tissues of cardiovascular safety concern. Although only two examples of dosing regimens involving a combination of loading dose and daily maintenance dose are provided in Table 3, it is not intended to limit the dosing regimens of this invention only to those provided in Table 3. Obviously, variations in the loading dose and consequent the daily maintenance dose levels may be conceived depending on indications of subjects. For example a loading dose of 2 mg may be used in combination with a daily maintenance dose of about 0.25 mg/day.

TABLE 3
Dosing regimens for Compound A which adopt loading dose
for the first day and daily maintenance dose in order
to achieve fast onset of the therapeutic activity while
keeping the convenience of daily once dosing.

| | | Daily Maintenance |
|---|---|---|
| Regimen | Loading Dose for Day 1 | Dose from Day 2 |
| A | 3 mg Compound A | 0.4 mg/day Compound A |
| B | 6 mg Compound B | 0.8 mg/day Compound A |

### Pharmacokinetic Observations in Healthy Male Subjects (Multiple Dose)

The dosing regimens of the instant invention were tested in healthy human male subjects. Table 4 provides mean pre-dose plasma levels of Compound A following oral administrations of capsule drug products containing Compound A according to either Regimen A or Regimen B of Table 3 daily once for 8 days. In both dosing regimens, the pre-dose plasma levels remained almost constant. Also there were no suggestions of drug accumulation from the multiple dosings. The plasma levels then decayed following the last dosing with expected mean elimination half-lives of 4 to 5 days for Regimen A and Regimen B. In conclusion, the pharmacokinetic outcomes from the tested multiple dosing regimens indicate that plasma levels of Compound A may be controlled essentially to constant levels to improve the cardiovascular safety over the dosing period without inducing drug accumulation upon repeat dosing.

TABLE 4
Pre-dose mean plasma concentrations of Compound A in healthy male
subjects who received capsule drug products containing Compound
A by either Regimen A or Regimen B of Table 3 for 8 days.

| | | Mean Pre-dose Plasma Level, ng/ml | |
|---|---|---|---|
| Daily Event | Day | Regimen A | Regimen B |
| Loading Dose | 1 | 0 | 0 |
| Maintenance Dose | 2 | 3.8 | 6.2 |
| | 3 | 4.2 | 6.2 |
| | 4 | 4.0 | 6.3 |
| | 5 | 4.7 | 6.5 |
| | 6 | 4.5 | 6.3 |
| | 7 | 3.6 | 6.6 |
| | 8 | 3.6 | 7.1 |
| No Dose | 9 | 4.0 | 6.6 |

1. A method to treat a COX-2 mediated disorder by administering to a subject Compound A, or a pharmaceutically acceptable salt or composition thereof according to a dosing regimen adopting a combination of loading dose and daily maintenance dose so as to maintain an essentially constant therapeutic plasma level of Compound A:

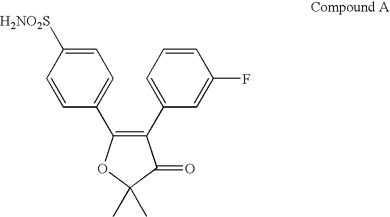

2. Within claim 1, a method to treat a COX-2 mediated disorder by administering to a subject Compound A, or a pharmaceutically acceptable salt or composition thereof according to a dosing regimen adopting a combination of loading dose and daily maintenance dose so as to maintain an essentially constant therapeutic plasma level of Compound A, wherein the loading dose is up to 15 mg and the daily maintenance dose is not more than 20% of the loading dose per day.

3. Within claim 1, a method to treat a COX-2 mediated disorder requiring a long term treatment by administering to a subject Compound A, or a pharmaceutically acceptable salt or composition thereof according to a dosing regimen adopting a combination of loading dose and daily maintenance dose so as to maintain an essentially constant therapeutic plasma level of Compound A.

4. Within claim 2, a method to treat a COX-2 mediated disorder requiring a long term treatment by administering to a subject Compound A, or a pharmaceutically acceptable salt or composition thereof according to a dosing regimen adopting a combination of loading dose and daily maintenance dose so as to maintain an essentially constant therapeutic plasma level of Compound A, wherein the loading dose is up to 15 mg and the daily maintenance dose is not more than 20% of the loading dose per day.
The present invention relates to a potent cyclooxygenase-2 inhibitor and dosing regimens thereof to safely treat cyclooxygenase-2 mediated disorders.
